(19) United States
(12) Patent Application Publication
(10) Pub. No.: US 2018/0318003 A1
(43) Pub. Date: Nov. 8, 2018

(54) DEVICES AND METHODS FOR MYOCARDIAL REDUCTION THERAPY

(71) Applicant: Mayo Foundation for Medical Education and Research, Rochester, MN (US)

(72) Inventors: Ammar M. Killu, Rochester, MN (US); Elad Maor, Rochester, MN (US); Rick A. Nishimura, Rochester, MN (US); Samuel J. Asirvatham, Rochester, MN (US); Amir Lerman, Rochester, MN (US)

(73) Assignee: Mayo Foundation for Medical Education and Research, Rochester, MN (US)

(21) Appl. No.: 15/968,383

(22) Filed: May 1, 2018

Related U.S. Application Data
(60) Provisional application No. 62/501,408, filed on May 4, 2017.

(51) Int. Cl. A61B 18/14

(52) U.S. Cl. CPC A61B 18/1492; A61B 2018/00577

(57) ABSTRACT

Devices and methods can be used to treat heart conditions such as hypertrophic cardiomyopathy (HCM). For example, this document describes transcatheter myocardial volume reduction devices and methods for treating HCM. In some implementations described in this document, a transcatheter irreversible electroporation (IRE) technique is used to treat HCM. In some cases, such a technique is used to deliver non-thermal ablation to myocardial cellular components such as the ventricular septum. The bulk of the ventricular septum's myocardial tissue can be reduced in result.

### CROSS-REFERENCE TO RELATED APPLICATIONS

This application claims the benefit of U.S. Provisional Application Ser. No. 62/501,408, filed May 4, 2017. The disclosure of the prior application is considered part of (and is incorporated by reference in) the disclosure of this application.

### BACKGROUND

### 1. Technical Field

This document relates to devices and methods for treating heart conditions. For example, this document relates to transcatheter myocardial volume reduction devices and methods for treating hypertrophic obstructive cardiomyopathy

### 2. Background Information

Hypertrophic cardiomyopathy (HCM) is a congenital disease in which the heart muscle (myocardium) becomes abnormally thick (hypertrophied). The thickened heart muscle can make it harder for the heart to pump blood.

About one in 500 people across all populations have HCM. HCM is a genetic predisposition. The children of a parent with HCM have a 50/50 chance of inheriting HCM from the parent.

HCM often goes undiagnosed because people with the disease may have few, if any, symptoms and can lead normal lives with no significant problems. However, in some people with HCM, the thickened heart muscle can cause shortness of breath, chest pain or problems in the heart's electrical system, potentially resulting in life-threatening abnormal heart rhythms (arrhythmias).

### SUMMARY

This document describes devices and methods for treating heart conditions. For example, this document describes transcatheter myocardial volume reduction devices and methods for treating HCM.

In one aspect, this disclosure is directed to a method of reducing a volume of myocardial tissue in a heart. The method includes: positioning a first electrode in a right ventricle of the heart and adjacent a ventricular septal wall; positioning a second electrode in a left ventricle of the heart and adjacent the ventricular septal wall; and delivering a pulsed electrical field from the first electrode to the second electrode through the ventricular septal wall.

Such a method of reducing a volume of myocardial tissue in a heart may optionally include one or more of the following features. The delivering the pulsed electrical field may induce electroporation of cells of the ventricular septal wall. The electroporation of the cells may result in a reduced volume of myocardial tissue in the ventricular septal wall. The electroporation of the cells may comprise irreversible electroporation.

In another aspect, this disclosure is directed to a method of reducing a volume of myocardial tissue in a heart. The method includes: positioning an array of first electrodes in a right ventricle of the heart and adjacent a ventricular septal wall; positioning an array of second electrodes in a left ventricle of the heart and adjacent the ventricular septal wall; and delivering a pulsed electrical field from the first electrodes to the second electrodes through the ventricular septal wall.

Such a method of reducing the volume of myocardial tissue in the heart may optionally include one or more of the following features. The delivering the pulsed electrical field may induce electroporation of cells of the ventricular septal wall. The electroporation of the cells may result in a reduced volume of myocardial tissue in the ventricular septal wall. The electroporation of the cells may comprise irreversible electroporation. The method may also include adjusting the size or shape of the array of first electrodes or the array of second electrodes.

Particular embodiments of the subject matter described in this document can be implemented to realize one or more of the following advantages. In some embodiments, heart conditions such as HCM and others can be treated using the devices and methods provided herein. The volume of the ventricular septum's myocardial tissue can be advantageously reduced in result. Accordingly, the symptoms of HCM may also be reduced. For example, in some cases tissue volume reduction can relieve an obstruction of a heart valve, decrease a pressure gradient within or near a heart valve, increase the volume of a heart chamber, and/or increase the stroke volume of a heart. In some cases, non-thermal IRE can be induced within microseconds, without generation of heat that could potentially damage extra cellular components. In some embodiments, HCM can be treated in a minimally invasive fashion using the devices and methods provided herein. Such minimally invasive techniques can reduce recovery times, patient discomfort, and treatment costs. In some embodiments, HCM can be treated in time efficient manner using the devices and methods provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is an illustration of a normal heart.

FIG. 2 is an illustration of a heart with hypertrophic cardiomyopathy (HCM).

FIG. 3 is a fluoroscopic image from a right anterior oblique viewpoint of a heart having a first electrode device in the right ventricle and a second electrode device in the left ventricle.

FIG. 4 is a fluoroscopic image from a left anterior oblique viewpoint of a heart having a first electrode device in the right ventricle and a second electrode device in the left ventricle.

Like reference numbers represent corresponding parts throughout.

### DETAILED DESCRIPTION

This document describes devices and methods for treating heart conditions. For example, this document describes transcatheter myocardial volume reduction devices and methods for treating HCM. In some implementations described in this document, a transcatheter irreversible electroporation (IRE) technique is used to treat HCM. In some cases, such a technique is used to deliver non-thermal ablation to myocardial cellular components, such as of the ventricular septum. The volume of the ventricular septum's myocardial tissue can be reduced in result. Accordingly, the symptoms of HCM may also be reduced. For example, in some cases tissue volume reduction can relieve an obstruction of a heart valve, reduce diastolic dysfunction, decrease a pressure gradient within or near a heart valve, increase the volume of a heart chamber, and/or increase the stroke volume of a heart.

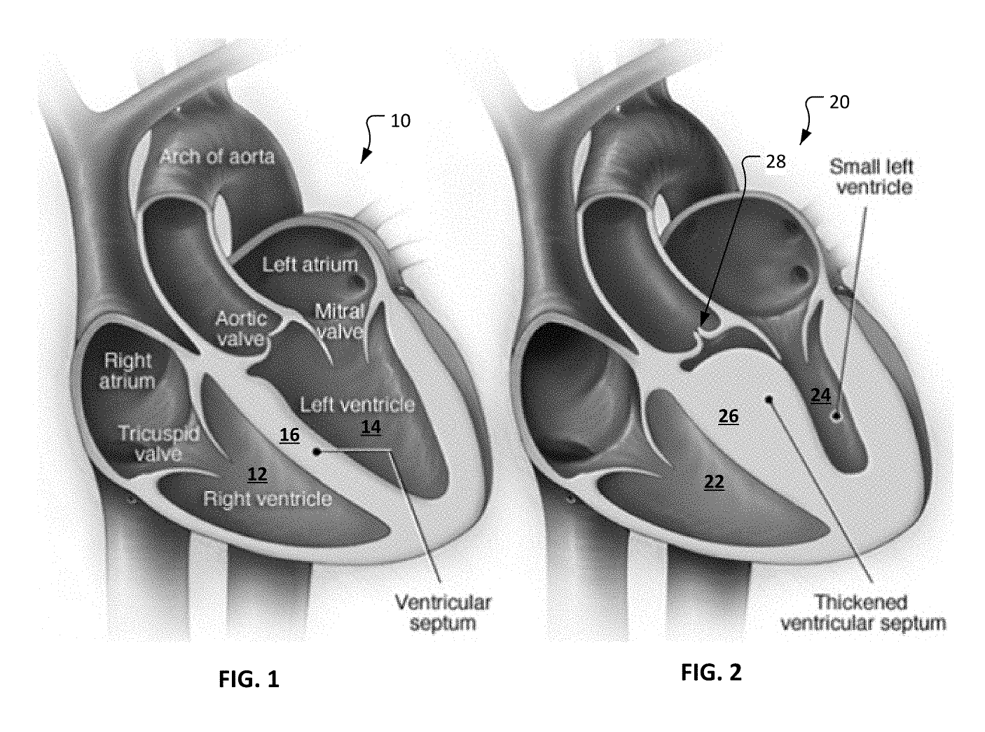

FIG. 1 shows a schematic cross-section of a normal, healthy human heart 10. Of note in this context are the right ventricle 12, left ventricle 14, and ventricular septum 16. Ventricular septum 16 has a normal wall thickness in FIG. 1.

FIG. 2, in contrast, shows a schematic cross-section of a heart 20 that has an HCM condition. It can be seen that the heart walls (muscle) are much thicker (hypertrophied) in the HCM heart 20. That is, in particular it can be seen that ventricular septum 26 (which separates right ventricle 22 and left ventricle 24) has a wall thickness that is thicker than the normal wall of ventricular septum 16 (FIG. 1). If fact, it can be seen in FIG. 2 that the thickness of ventricular septum 26 encroaches on the outflow tract of aortic valve 28.

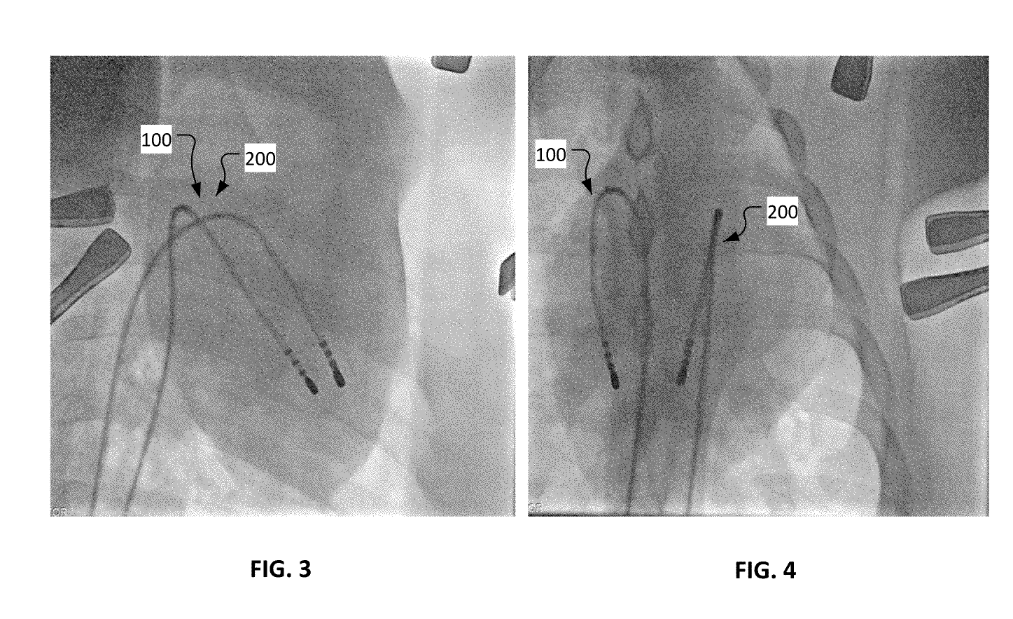

Referring also to FIGS. 3 and 4, the devices and methods described herein can be used to treat HCM. For example, the devices and methods described herein can be used to reduce the thickness of ventricular septum 26 by using two transcatheter unipolar electrode devices 100 and 200 to deliver non-thermal IRE to ventricular septum 26. In one such example, a first transcatheter unipolar electrode device 100 can be positioned in right ventricle 22 and a second transcatheter unipolar electrode device 200 can be positioned in left ventricle 24. The first and second transcatheter electrode devices 100 and 200 can be positioned adjacent to the wall of ventricular septum 26. Accordingly, the delivery of a pulsed electrical field between the first unipolar electrode 100 and the second unipolar electrode 200 through ventricular septum 26 will cause the non-thermal IRE to the myocardium of ventricular septum 26.

Non-thermal IRE involves the delivery of electroporation energy which induces cell death by creating pores in cell membranes that are sufficient, for example, to cause irreversible loss and/or imbalance of intracellular components. In some cases, IRE can be induced within microseconds, without generation of heat that could potentially damage extra cellular components.

Delivery of non-thermal IRE using the electrodes 100 and 200, in some cases, causes some of the tissue comprising the tissue bulk (e.g., ventricular septum 26) to be initially inactivated. Over a period of time (e.g., a few days to a week), some cellular material in the tissue dissolves, resulting in a thinning of ventricular septum 26 (or other targeted tissue). In some implementations, additionally or alternatively, the targeted tissue is a wall of right ventricle 22 or left ventricle 24.

In some implementations, the transcatheter unipolar electrode devices 100 and 200 comprise a single catheter shaft on which one or more electrodes are disposed. In some implementations, the transcatheter electrode devices 100 and 200 comprise an expandable framework on which an array of electrodes are disposed. In some such implementations, the area and or shape of the expandable framework is selectively adjustable by a clinician to conform to a particular anatomy or usage.

In addition to the treatment of HCM as described herein, similar techniques and systems of one or more devices can be used to improve other myocardial performance aspects. For example, in some cases electroporation can be used to improve myocardial performance in the following manner. In some embodiments, a first device with one or more electrodes (e.g., a first set of anodes) is placed endocardially, and a second device with one or more electrodes (e.g., a second set of cathodes) is placed in the pericardial space or the coronary vasculature. Bipolar electroporation can be delivered using the first and second electrode devices.

Contemporaneous with the delivery of the electroporation, the transmyocardial impedance can be measured. The transmyocardial impedance measurements can be used as a surrogate for contractility as well as lesion formation along with other direct measures of myocardial performance, such as dP/dt or tissue Doppler imaging done via appropriate sensors mounted along the electrodes. Accordingly, systolic and/or diastolic myocardial performance can be measured and used as a feedback loop, and the electroporation energy delivery can be optimized to either maximize systolic performance and/or improve diastolic performance of the ventricular myocardium.

Additional treatment modalities that are effective for treating other cardiac conditions are also envisioned within the scope of this disclosure. For example, in some embodiments bipolar electroporation (as well as widely-spaced-bipolar and monopolar electroporation) can be delivered using appropriately placed electrodes including those in a fixed pattern mounted on a single but two-armed device for local myocardial resection. For example, in some cases such a device and technique can be used as a treatment for myocardial bridges where the electrodes may be in the vasculature as well as the endocardial surface and epicardial surface of the heart. In additional examples, conditions such as, but not limited to, cardiac tumors, localized hypertrophy, noncompaction syndrome, and hypertrabeculations, can be treated similarly. In addition to the ventricular myocardial alteration techniques described herein, the electroporation delivery device and circuit and feedback algorithm may be used to modify the endothelial surface(s) such as over valves, for example, when calcified, fibrotic, and/or when the neural endocardium is pathological such as with endomyocardial fibroelastosis. Similarly, the electroporation devices with electrodes as described herein can be positioned either wholly or partly in the cardiac vasculature and epicardial surface. In that fashion, the devices may deliver electroporation to treat inflammatory disorders including infectious disorders of the endothelium, such as endocarditis and infected prosthetic devices, as well as noninfectious inflammatory processes.

While, as described herein, the primary intent and specific energy and field creation algorithms of this disclosure target myocardial modulation, an inverse application is also envisioned that is specifically for myocardial sparing and/or modulation of the sensory nerves of the heart as a treatment for intractable cardiac pain syndromes.

### Examples

METHODS: A study was performed to confirm the efficacy of using two electrode devices (one each in the right and left ventricles) to deliver non-thermal IRE to reduce myocardial tissue volume. Two 30 kg pigs were used in this chronic study. Under general anesthesia, femoral vascular access was obtained. BLAZER™ catheters (Boston Scientific, MA) were introduced into the left and right ventricles using fluoroscopy and intracardiac ultrasound (ICE) guidance (FIGS. 3 and 4). IRE was performed by applying 10 direct-current electric pulses of 100 microseconds duration. First treatment included pulses of 1,000 Volts that were delivered with ECG-gating using a clinical pulse generator (NANOKNIFE®, Angiodynamics, NY). If tolerated well, second treatment included pulses of 2,000 V that were delivered in a similar way. Overall therapy lasted less than 1 minute.

The acute effect of IRE was evaluated using ICE and intracardiac electrograms. At 28 days, the chronic effect of IRE was evaluated by in-vivo ICE, in-vivo intracardiac electrograms, as well as by post-mortem gross pathology and histology.

RESULTS: IRE ablation was uneventful in both animals. Immediately following IRE, local electrograms showed mitigation of near field signals acutely and at a similar location chronically. Intracardiac ultrasound showed wall motion abnormality and acute edema immediately following the procedure, as well as thinning of the ventricular septum at 28 days. Gross pathology demonstrated deep myocardial lesion at the sites of IRE, with more than 50% reduction in ventricular wall thickness at 28 days. The left anterior descending artery was not damaged despite its proximity to the ablated zone. Histology of the treated segments shows areas of fibrous connective tissue beneath the epicardial surface with myocyte degeneration.

DISCUSSION: A finding of this experiment is that IRE can be delivered with a transcatheter approach using two electrode catheters (one each in the right and left ventricles) to create deep chronic myocardial ablation lesions in the ventricular septum. Lavee and colleagues used an in-vivo open heart porcine model to show how epicardial IRE pulses induce ablation of atrial tissue. Their protocol utilized a sequence of 8, 16, or 32 direct-current pulses of 1500 to 2000 Volts, 100 microsseconds each, at a frequency of 5 per second, applied between two parallel electrodes. Wittkampf and colleagues demonstrated how epicardial IRE can induce deep myocardial lesions without affecting adjacent coronary arteries. In their studies, they used a monophasic external defibrillator to apply 50, 100 or 200 Joule pulses. The results of this experimentation extend these previous observations.

The experiment showed that electroporation pulses can be delivered by two electrode devices (one in the right ventricle and another in the left ventricle) in a transcatheter approach to induce non-thermal ablation of the ventricular septum. The experiment's chronic model demonstrated that the lesion was associated with sustained mitigation of near field signals at 28 days, as well as more than 50% reduction in the thickness of the ventricular septum. IRE is not associated with thermal denaturation of proteins and preserves extra cellular components. With these properties, IRE lends itself to being suitable candidate for multiple clinical applications. First, for ablation of deep ventricular arrhythmogenic foci. Second, for a safe ablation approach for posterior left atrial structures without the risk of esophageal thermal damage. Last, by targeting the ventricular septum in a trans-catheter approach, it holds the potential to treat hypertrophic obstructive cardiomyopathy.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method of reducing a volume of myocardial tissue in a heart, the method comprising:
positioning a first electrode in a right ventricle of the heart and adjacent a ventricular septal wall;
positioning a second electrode in a left ventricle of the heart and adjacent the ventricular septal wall; and
delivering a pulsed electrical field from the first electrode to the second electrode through the ventricular septal wall.

2. The method of claim 1, wherein the delivering the pulsed electrical field induces electroporation of cells of the ventricular septal wall.

3. The method of claim 2, wherein the electroporation of the cells results in a reduced volume of myocardial tissue in the ventricular septal wall.

4. The method of claim 2, wherein the electroporation of the cells comprises irreversible electroporation.

5. A method of reducing a volume of myocardial tissue in a heart, the method comprising:
positioning an array of first electrodes in a right ventricle of the heart and adjacent a ventricular septal wall;
positioning an array of second electrodes in a left ventricle of the heart and adjacent the ventricular septal wall; and
delivering a pulsed electrical field from the first electrodes to the second electrodes through the ventricular septal wall.

6. The method of claim 5, wherein the delivering the pulsed electrical field induces electroporation of cells of the ventricular septal wall.

7. The method of claim 6, wherein the electroporation of the cells results in a reduced volume of myocardial tissue in the ventricular septal wall.

8. The method of claim 6, wherein the electroporation of the cells comprises irreversible electroporation.

9. The method of claim 5, further comprising adjusting the size or shape of the array of first electrodes or the array of second electrodes.
